(19) United States
(12) Patent Application Publication
(10) Pub. No.: US 2026/0242331 A1
(43) Pub. Date: Aug. 20, 2026

(54) COMPOUND AND USE THEREOF IN IDENTIFICATION OF CAMELLIA PTILOPHYLLA VARIETIES

(71) Applicants: TEA RESEARCH INSTITUTE, GUANGDONG ACADEMY OF AGRICULTURAL SCIENCES, Guangzhou (CN); SOUTH CHINA BOTANICAL GARDEN, CHINESE ACADEMY OF SCIENCES, Guangzhou (CN)

(72) Inventors: Kaixing FANG, Guangzhou (CN); Hualing WU, Guangzhou (CN); Shengxiang QIU, Guangzhou (CN); Shulin DENG, Guangzhou (CN); Hongjian LI, Guangzhou (CN); Mingxing XU, Guangzhou (CN); Dandan QIN, Guangzhou (CN); Erdong NI, Guangzhou (CN); Chendong PAN, Guangzhou (CN); Bo LI, Guangzhou (CN); Qing WANG, Guangzhou (CN); Qiushuang WANG, Guangzhou (CN); Xiaohui JIANG, Guangzhou (CN)

(21) Appl. No.: 19/161,466

(22) PCT Filed: Apr. 10, 2024
(86) PCT No.: PCT/CN2024/087018

§ 371 Date: Sep. 2, 2025

(30) Foreign Application Priority Data
Apr. 13, 2023 (CN) 2023103975820

(51) Int. Cl. C07C 229/22; G01N 30/02; G01N 33/68

(52) U.S. Cl. CPC C07C 229/22; G01N 30/02; G01N 33/6812; G01N 2030/027; G01N 2333/415

(57) ABSTRACT

The present disclosure relates to a compound and use thereof in an identification of Camellia ptilophylla varieties, with a structural formula of Formula (I). Upon identification, the compound was named as 4-amino-5-hydroxypentanoic acid, being a newly discovered natural compound found in tender shoots of Camellia ptilophylla, and also identified as a novel amino acid. This compound was not detected in other tea varieties, but it was consistently present in all populations of Camellia ptilophylla. In the tender shoot tissues or tea products made from such tender shoot materials, the detection of this compound can effectively identify tea products and seedlings of Camellia ptilophylla.

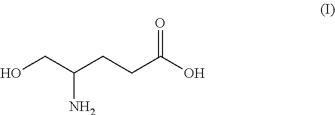

### TECHNICAL FIELD

The present disclosure relates to the technical field of tea plant varieties and tea product identification, and specifically, to a compound and use thereof in an identification of Camellia ptilophylla varieties.

### BACKGROUND

Chinese tea plant resources are widely distributed and diverse in types. Most of the wild tea plant germplasms are distributed in the form of communities. Through years of natural and artificial selection, they have developed rich variations, forming a plurality of species and varieties. Thus, tea plants constitute a vast family. Drinking tea can refresh the mind due to the presence of caffeine in tea leaves. However, certain groups of people, including pregnant women, infants and young children, individuals with poor sleep, and those with hypoglycemia, should avoid excessive caffeine intake.

Camellia ptilophylla is a rare tea germplasm resource discovered in Huizhou, Guangdong Province, with its buds and leaves used to produce various types of tea such as green tea, white tea, and black tea. It is renowned for its shoots containing little or no caffeine. Due to its low-caffeine characteristics, the products developed from Camellia ptilophylla also exhibit low-caffeine properties, making the development of Camellia ptilophylla tea products highly valuable in terms of both application and economic benefits.

The identification of Camellia ptilophylla tea products and seedlings can effectively regulate the purity of seed sources at the planting stage, while also enabling traceability of Camellia ptilophylla tea products to ensure their reliability.

Traditionally, the identification of Camellia ptilophylla seedlings or products can be carried out by detecting the caffeine content. However, there are other tea germplasm resources that exhibit low caffeine characteristics. Therefore, detection based on caffeine content alone cannot fully confirm whether the tea seedlings or tea products are derived from Camellia ptilophylla.

### SUMMARY

The present disclosure aims to overcome the above-mentioned deficiencies of the existing technology and to provide a compound and use thereof in an identification of Camellia ptilophylla tea. By detecting 4-amino-5-hydroxypentanoic acid in seedling tender shoots or tea products made from the tender shoots, the present disclosure enables the detection of Camellia ptilophylla seedlings or Camellia ptilophylla tea products.

The first objective of the present disclosure is to provide a compound.

The second objective of the present disclosure is to provide use of the compound in the identification of Camellia ptilophylla varieties.

The third objective of the present disclosure is to provide a method for identifying Camellia ptilophylla.

The fourth objective of the present disclosure is to provide a method for detecting a compound having a structural formula of Formula (I).

The fifth objective of the present disclosure is to provide use of a detection reagent for the compound in a preparation of a kit for identifying Camellia ptilophylla.

The sixth objective of the present disclosure is to provide a kit for detecting and identifying Camellia ptilophylla.

To achieve the above objectives, the present disclosure is implemented through the following solutions: by detecting 4-amino-5-hydroxypentanoic acid in seedling tender shoots or tea made from the tender shoots, the identification of Camellia ptilophylla seedlings or tea products can be achieved.

The present disclosure has discovered through research that 4-amino-5-hydroxypentanoic acid is a newly identified natural compound found in the tender shoots of Camellia ptilophylla, and it is also a newly discovered amino acid. It has not been detected in other tea varieties but can be detected in all populations of Camellia ptilophylla. Therefore, detecting this compound can be used to identify seedlings and plants of Camellia ptilophylla.

The present disclosure discloses a compound having a structural formula of Formula (I),

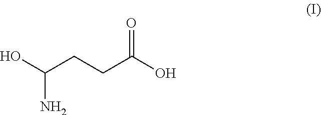

The present disclosure further discloses use of the compound in the identification of Camellia ptilophylla varieties.

Preferably, the compound is used in the identification of Camellia ptilophylla varieties for products and/or seedlings.

The present disclosure further discloses a method for identifying Camellia ptilophylla, including detecting the compound in a sample to be tested:
the sample to be tested is Camellia ptilophylla if the sample to be tested contains the compound.

Preferably, the sample to be tested is tender shoots of Camellia ptilophylla.

Preferably, high-performance liquid chromatography (HPLC) analysis is performed on a 2,4-dinitrofluorobenzene (DNFB) derivatization product of an aqueous extract of the sample to be tested.

Further disclosed is a method for detecting a compound having a structural formula of Formula (I), including performing the HPLC analysis on the DNFB derivatization product of the aqueous extract of the sample to be tested.

More preferably, a method for preparing the aqueous extract of the sample to be tested is as follows: the sample to be tested is mixed with boiling water, fully extracted under a boiling condition, and a solid is removed to obtain the aqueous extract.

Further preferably, when the sample to be tested is in powder form, a ratio of the sample to be tested to the aqueous extract is 1 g: 80 mL to 120 mL, with an extraction time of 30 min to 50 min.

Further preferably, for the sample to be tested in powder form, the ratio of the sample to be tested to the aqueous extract is 1 g: 100 mL, with the extraction time of 45 min.

Further preferably, when the sample to be tested is a steamed green sample, the ratio of the sample to be tested to the aqueous extract is 1 kg: 3 L to 5 L, with the extraction time of 1 h to 3 h.

Further preferably, for the steamed green sample, the ratio of the sample to be tested to the aqueous extract is 1 kg: 4 L, with the extraction time of 2 h.

Preferably, a derivatization method is as follows: a mixture of the aqueous extract of the sample to be tested, an acetonitrile solution of 2,4-dinitrofluorobenzene and a NaHCO₃ buffer solution undergoes a derivatization reaction in the dark at 50° C. to 70° C. to yield a derivatized product.

More preferably, the derivatization reaction is continued at 60° C. in the dark for no less than 1 h.

More preferably, the mixture is a combination of the aqueous extract of the sample to be test, an 8 mL/L to 12 mL/L acetonitrile solution of 2,4-dinitrofluorobenzene, and a 0.4 mol/L to 0.6 mol/L NaHCO₃ buffer solution (pH 8.5 to 9.5) at a volume ratio of 1 to 10:1 to 10:1 to 1.

Most preferably, the mixture is a combination of the aqueous extract of the sample to be test, a 10 mL/L acetonitrile solution of 2,4-dinitrofluorobenzene, and a 0.5 mol/L NaHCO₃ buffer solution (pH 9.0) at a volume ratio of 10:10:1.

More preferably, after the derivatization reaction, purification is performed followed by HPLC analysis.

Most preferably, the purification is performed by filtration through a 0.45 μm filter.

Preferably, the chromatographic column is an Eclipse Plus C18 column, and the mobile phase consists of mobile phase A and mobile phase B, wherein the mobile phase A is a NaAc buffer solution containing N,N-dimethylformamide, and the mobile phase B is an acetonitrile aqueous solution, performing a gradient elution.

More preferably, a temperature of the chromatographic column is 26° C. to 30° C.

Most preferably, the temperature of the chromatographic column is 28° C.

More preferably, a detection wavelength is 300 nm to 420 nm in the high-performance liquid chromatography analysis.

Most preferably, the detection wavelength is 360 nm in the high-performance liquid chromatography analysis.

More preferably, the mobile phase A is a NaAc buffer solution containing 8 mL/L to 12 mL/L of N,N-dimethylformamide, with a pH of 6.0 to 7.0 and a concentration of 0.04 mol/L to 0.06 mol/L.

Further preferably, the mobile phase A is a NaAc buffer solution containing 10 mL/L of N,N-dimethylformamide, with a pH of 6.5 and a concentration of 0.05 mol/L.

More preferably, the mobile phase B is an acetonitrile aqueous solution with a volume ratio (V/V) of 1 to 2:1 to 2.

Further preferably, the mobile phase B is an acetonitrile aqueous solution with a V/V of 1:1.

As a specific embodiment of the present disclosure, the detailed method for HPLC analysis is as follows:
the chromatographic column is an Eclipse Plus C18 column, and the mobile phase consists of mobile phase A and mobile phase B, where the mobile phase A is a 0.05 mol/L NaAc buffer solution containing 10 mL/L N,N-dimethylformamide with a pH of 6.5, and the mobile phase B is an acetonitrile aqueous solution with a V/V ratio of 1:1.

The gradient elution program is calculated by volume ratio as follows:
0 to 6 min: the mobile phase A 100% and the mobile phase B 0%;
6 to 12 min: the mobile phase A 90% and the mobile phase B 10%;
12 to 18 min: the mobile phase A 85% and the mobile phase B 15%;
18 to 33 min: the mobile phase A 80% and the mobile phase B 20%;
33 to 42 min: the mobile phase A 75% and the mobile phase B 25%;
42 to 50 min: the mobile phase A 70% and the mobile phase B 30%;
50 to 55 min: the mobile phase A 50% and the mobile phase B 50%;
55 to 60 min: the mobile phase A 20% and the mobile phase B 80%;
60 to 65 min: the mobile phase A 0% and the mobile phase B 100%;
65 to 70 min: the mobile phase A 0% and the mobile phase B 100%;
70 to 80 min: the mobile phase A 100% and the mobile phase B 0%.

The column temperature is set at 28° C., the detection wavelength is set at 360 nm, the mobile phase flow rate is 1.0 mL/min, and the sample injection volume is 5 μL. A characteristic peak appears at 42 min, indicating the presence of the compound having the structural formula of Formula (I).

The present disclosure further discloses use of the detection reagent for the compound in the preparation of a kit for identifying Camellia ptilophylla.

The present disclosure further discloses a kit for detecting and identifying Camellia ptilophylla, including the detection reagent of the compound.

Preferably, the detection reagent is a derivatization reagent and/or an HPLC analysis reagent.

More preferably, the derivatization reagent is an acetonitrile solution of DNFB and a NaHCO₃ buffer solution.

More preferably, the acetonitrile solution of DNFB is an 8 mL/L to 12 mL/L DNFB acetonitrile solution.

Further preferably, the acetonitrile solution of DNFB is a 10 mL/L DNFB acetonitrile solution. More preferably, the NaHCO₃ buffer solution is a 0.4 mol/L to 0.6 mol/L NaHCO₃ buffer solution at pH of 8.5 to 9.5.

Further preferably, the NaHCO₃ buffer solution is a 0.5 mol/L NaHCO₃ buffer solution at pH 9.0.

More preferably, the HPLC analysis reagent includes a mobile phase, which includes mobile phase A and mobile phase B, where the mobile phase A is a NaAc buffer solution containing N,N-dimethylformamide, and the mobile phase B is an acetonitrile aqueous solution.

More preferably, the mobile phase A is a NaAc buffer solution containing 8 mL/L to 12 mL/L of N,N-dimethylformamide, with a pH of 6.0 to 7.0 and a concentration of 0.04 M to 0.06 M.

More preferably, the mobile phase A is a NaAc buffer solution containing 10 mL/L of N,N-dimethylformamide, with a pH of 6.5 and a concentration of 0.05 mol/L.

More preferably, the mobile phase B is an acetonitrile aqueous solution with a V/V of 1 to 2:1 to 2.

Still more preferably, the mobile phase B is an acetonitrile aqueous solution with a V/V of 1:1. Compared with the existing technology, the present disclosure has the following beneficial effects:

4-amino-5-hydroxypentanoic acid is a newly discovered natural compound found in tender shoots of Camellia ptilophylla, and it is also a newly identified amino acid. This compound has not been detected in other tea varieties but can be found in all populations of Camellia ptilophylla. In the shoot tissues or in tea products made from shoot materials, the detection of this compound can effectively identify tea products and seedlings of Camellia ptilophylla.

### BRIEF DESCRIPTION OF THE DRAWINGS

FIG. 1 shows the amino acid components of different tea types in Camellia ptilophylla populations and Ying Hong No. 9, where Ala is alanine, Arg is arginine, Asp is aspartic acid, Cys-cys is cysteine, Glu is glutamic acid, Gly is glycine, His is histidine, Ile is isoleucine, Leu is leucine, Lys is lysine, Met is methionine, Phe is phenylalanine, Ser is serine, The is theanine, Thr is threonine, Tyr is tyrosine, and Val is valine.

FIG. 2 shows the total amino acids in different tea types of Camellia ptilophylla populations and Yinghong No. 9.

FIG. 3 shows the amino acid components in tender shoots and roots of Camellia ptilophylla. Panel A of FIG. 3 shows 35 amino acid components in fresh leaves of Camellia ptilophylla and other tea resources. Panel B of FIG. 3 shows the theanine detection chromatogram in root systems of Camellia ptilophylla A35. Panel C of FIG. 3 shows the detection chromatogram of theanine in the root systems of Camellia oleifera. Panel D of FIG. 3 shows the detection chromatogram of theanine in the root systems of Yinghong No. 9. Panel E of FIG. 3 shows the theanine standard.

FIG. 4A-FIG. 4B show the amino acid profile characteristics of different tissue parts in Camellia ptilophylla No. 1 and Ying Hong No. 9. FIG. 4A shows the sampling standards for different tissue parts, where 1-9 represent the bud, first leaf, second leaf, upper stem segment, lower stem segment, mature leaf, root system, flower bud, and fruit, respectively. FIG. 4B shows the amino acid determination profiles of Camellia ptilophylla No. 1 and Yinghong No. 9, where a1-a9 and b1-b9 represent different tea plant tissues of Camellia ptilophylla No. 1 and Yinghong No. 9, respectively. The black arrow represents unknown amino acids in Camellia ptilophylla, while the red arrow indicates theanine in Camellia sinensis.

FIG. 5 shows the content of different amino acids in various tissue parts of three Camellia ptilophylla lines and three tea varieties during spring. Panels A-S of FIG. 5 represent the contents of total amino acids (Taa), theanine (The), aspartic acid (Asp), glutamic acid (Glu), arginine (Arg), serine (Ser), phenylalanine (Phe), threonine (Thr), lysine (Lys), alanine (Ala), isoleucine (Ile), tyrosine (Tyr), valine (Val), methionine (Met), histidine (His), cystine (Cys-cys), glycine (Gly), leucine (Leu), and unknown amino acids in different tissue parts of six varieties, respectively. Here, CTN1 is Camellia ptilophylla No. 1, CTN2 is Camellia ptilophylla No. 2, CTN3 is Camellia ptilophylla No. 3, ZJ is Zijuan, YH9 is Yinghong No. 9, and YH12 is Hongyan No. 12. Bud refers to the bud. First leaf refers to the first leaf of the tender shoot. Second leaf refers to the second leaf of the tender shoot. Mature leaf refers to the old leaf on the semi-lignified stem. Stem 1 refers to the upper section of the tender stem. Stem 2 refers to the lower section of the tender stem; and Root refers to the fibrous root.

FIG. 6 shows the contents of different amino acids in various tissues of three Camellia ptilophylla strains and three tea varieties during summer. Panels A-S of FIG. 6 represent the contents of total amino acids (Taa), theanine (The), aspartic acid (Asp), glutamic acid (Glu), arginine (Arg), serine (Ser), phenylalanine (Phe), threonine (Thr), lysine (Lys), alanine (Ala), isoleucine (Ile), tyrosine (Tyr), valine (Val), methionine (Met), histidine (His), cystine (Cys-cys), glycine (Gly), leucine (Leu), and unknown amino acids (unknown) in different tissue parts of six varieties, respectively. Here, CTN1 is Camellia ptilophylla No. 1, CTN2 is Camellia ptilophylla No. 2, CTN3 is Camellia ptilophylla No. 3, ZJ is Zijuan, YH9 is Yinghong No. 9, and YH12 is Hongyan No. 12. Bud refers to the bud. First leaf refers to the first leaf of the tender shoot. Second leaf refers to the second leaf of the tender shoot. Mature leaf refers to the old leaf on the semi-lignified stem. Stem 1 refers to the upper section of the tender stem. Stem 2 refers to the lower section of the tender stem; and Root refers to the fibrous root.

FIG. 7 shows the content of different amino acids in various tissues of three Camellia ptilophylla strains and three tea varieties during autumn. Panels A-S of FIG. 7 represent the contents of total amino acids (Taa), theanine (The), aspartic acid (Asp), glutamic acid (Glu), arginine (Arg), serine (Ser), phenylalanine (Phe), threonine (Thr), lysine (Lys), alanine (Ala), isoleucine (Ile), tyrosine (Tyr), valine (Val), methionine (Met), histidine (His), cystine (Cys-cys), glycine (Gly), leucine (Leu), and unknown amino acids (unknown) in different tissue parts of six varieties, respectively. CTN1 is Camellia ptilophylla No. 1, CTN2 is Camellia ptilophylla No. 2, CTN3 is Camellia ptilophylla No. 3, ZJ is Zijuan, YH9 is Yinghong No. 9, and YH12 is Hongyan No. 12. Bud refers to the bud. First leaf refers to the first leaf of the tender shoot. Second leaf refers to the second leaf of the tender shoot. Mature leaf refers to the old leaf on the semi-lignified stem. Stem 1 refers to the upper section of the tender stem. Stem 2 refers to the lower section of the tender stem; and Root refers to the fibrous root.

FIG. 8 shows the pairwise correlation between total amino acids, theanine, glutamic acid, and unknown amino acids in different seasons. Panel A of FIG. 8 shows the pairwise correlations between total amino acids, theanine, glutamic acid, and unknown amino acids in spring. Panel B of FIG. 8 shows the pairwise correlations between total amino acids, theanine, glutamic acid, and unknown amino acids in summer. Panel C of FIG. 8 shows the pairwise correlations between total amino acids, theanine, glutamic acid, and unknown amino acids in autumn. Taa represents total amino acids, The represents theanine, Glu represents glutamic acid, and Unk represents the unknown amino acid in Example 3. CTN1 is Camellia ptilophylla No. 1, CTN2 is Camellia ptilophylla No. 2, CTN3 is Camellia ptilophylla No. 3, ZJ is Zijuan, YH9 is Yinghong No. 9, and YH12 is Hongyan No. 12.

FIG. 9 shows the content of the unknown amino acids in different tea plants; wherein C. ptilophylla H. T. Chang is a Camellia ptilophylla; C. sinensis (L.) O. Kuntze is a tea; C. sinensis var. assamica (Masters) Kitamura is an Assam tea; C. sinensis var. pubilimba Chang is a Baimao tea, C. assamica var. Kucha is a Kucha, and C. sinensis var. assamica cv. Hainan-dayezhong is a Hainan-dayezhong tea.

FIG. 10 shows one-dimensional proton nuclear magnetic resonance (1H-NMR) spectrum of the derivative of unknown amino acids in Example 3.

FIG. 11 shows the biological Correlation Spectroscopy (COSY) spectrum of unknown amino acids in Example 3.

FIG. 12 shows the Heteronuclear Single Quantum Coherence (HSQC) spectrum of the derivative of unknown amino acids in Example 3.

FIG. 13 shows the Distortionless Enhancement by Polarization Transfer (DEPT) spectrum of the derivative of unknown amino acids in Example 3.

FIG. 14 shows Carbon nuclear magnetic resonance (13C-NMR) spectrum of the derivative of unknown amino acids in Example 3.

FIG. 15 shows the heteronuclear multiple bond correlation (HMBC) spectrum (1) of the derivative of unknown amino acids in Example 3.

FIG. 16 shows the HMBC spectrum (2) of the derivative of unknown amino acids in Example 3.

FIG. 17 shows the HMBC spectrum (2) of the derivative of unknown amino acids in Example 3.

FIG. 18 shows the mass spectrometric identification of an unknown amino acid derivative (DNFB-4-amino-5-hydroxypentanoic acid) in Example 3 and the chemical structure verification of 4-amino-5-hydroxypentanoic acid. Panel A of FIG. 18 shows the high-resolution mass spectrometry identification of DNFB-4-amino-5-hydroxypentanoic acid. Panel B of FIG. 18 shows the HPLC analysis of Sample 1 (purified DNFB-4-amino-5-hydroxypentanoic acid obtained through isolation and purification). Panel C of FIG. 18 shows the HPLC chromatogram of Sample 2 (the product of 4-amino-5-hydroxypentanoic acid standard after DNFB derivatization). Panel D of FIG. 18 shows the HPLC chromatogram of a 1:1 mixture of Sample 1 and Sample 2. Panel E of FIG. 18 shows the HPLC chromatogram of a 1:1 mixture of Sample 2 and water.

### DETAILED DESCRIPTION

The present disclosure is further described in detail below in conjunction with the drawings and specific embodiments in the specification. The embodiments are provided solely to explain the present disclosure and are not intended to limit the scope of the present disclosure. The test methods used in the following examples are all conventional methods unless otherwise specified. The materials and reagents used, unless otherwise specified, are commercially available reagents and materials.

### Tea Germplasm Resource Materials

The tea germplasm resources used are preserved in the “Guangdong Tea Germplasm Resource Bank” maintained by the Tea Research Institute of the Guangdong Academy of Agricultural Sciences, located in Yingde City, Guangdong Province, China.

The tea germplasm resources involve a total of 400 accessions, all belonging to tea and its variants, including: 300 accessions of Camellia ptilophylla resources (Camellia ptilophylla H. T. Chang), 40 accessions of Chinese tea resources (Camellia sinensis (L.) O. Kuntze), 29 accessions of Assam tea resources (Camellia sinensis var. assamica (Masters) Kitamura), 19 accessions of Baimao tea resources (Camellia sinensis var. pubilimba Chang), 2 accessions of Kucha resources (Camellia assamica var. kucha), and 10 accessions of Hainan-dayezhong tea resources (Camellia sinensis var. assamica cv. Hainan-dayezhong).

Camellia assamica var. kucha and Camellia sinensis var. assamica cv. Hainan-dayezhong are both varieties of Assam tea. Among the Camellia ptilophylla resources No. 1, No. 2, and No. 3 are new strains, while the remaining 297 accessions are individual plants with robust growth.

All 100 tea germplasm resources, except for Camellia ptilophylla, are newly bred tea plant varieties or selected new strains.

Each new variety or strain is planted in three rows, randomly distributed in the resource nursery, with each row representing a biological replicate.

The materials from individual plants of Camellia ptilophylla are uniformly collected and then evenly divided into three parts, with each part serving as one biological replicate. All resources are managed according to the same cultivation and planting methods.

### Example 1: Analysis of Differences in Flavor and Amino Acid Components between Camellia ptilophylla Populations and Assam Tea Resource Ying Hong No. 9

### I. Experimental Methods

### 1. Trial Production of Tea

Using one bud with two leaves from summer shoots of Yinghong No. 9 and 300 individual plants of Camellia ptilophylla as raw materials, trial production of black tea, white tea, and green tea was conducted respectively.

Trial production of black tea: the raw material was withered at 35° C. until moisture content reached 60%, then rolled until the tea leaves were tightly curled, with tea juice extractable between fingers. After rolling, the leaves were fermented in a fermentation room until the aroma changed from grassy to floral and fruity. The fermented tea leaves were baked at 80° C. for 2.5 h.

Trial production of green tea: the raw material was withered at 35° C. until the moisture content reached 70%. The wilted leaves were then pan-fired in a tea-frying wok at 220° C. for fixation. When the moisture content of the fixed leaves dropped to around 58% and a floral-fruity aroma emerged, the tea leaves were removed and spread out to cool. Then, the tea leaves were rolled until they become moist and sticky, tightly curled into strips. The rolled tea leaves were baked at 80° C. for 2.5 h.

Trial production of white tea: the raw material was withered at 35° C. until the moisture content reached 30%, then dried at 30° C. until 90% dryness, followed by drying at 80° C. for 2.5 h.

Preparation of tea infusion: grind tea leaves into powder, weigh 1 g of the powder and transfer same into an Erlenmeyer flask, and add 90 mL of boiling water. Place the Erlenmeyer flask in a water bath at 100° C. and heat for 45 min. Filter the tea liquor to remove tea residues, then dilute the tea liquor to a final volume of 100 mL.

### 2. Sensory Evaluation of Tea

Sensory evaluation of tea was conducted in accordance with the “GB/T 23776-2009 Methodology for Sensory Evaluation of Tea”.

### 3. Determination of Total Free Amino Acids in Tea Infusion (Ninhydrin Colorimetric Method)

The determination of total amino acids was conducted in accordance with the National Standard of the People's Republic of China “GB/T 8314-2013 Tea—Determination of total free amino acids”.

### 4. Determination of Dry Matter Content

The determination of dry matter content referred to the National Standard of the People's Republic of China “GB/T 8303-2013 Tea—Preparation of ground test sample and determination of dry matter content”.

### 5. Determination Method for Amino Acid Components in Tea Infusion (OPA Derivatization Method)

(1) Respectively draw borate buffer solution, ultrapure water, OPA (o-phthalaldehyde, derivatization reagent), FMOC (9-fluorenylmethyloxycarbonyl, derivatization reagent), diluent (2 mL aqueous solution with 10 μL phosphoric acid added), ultrapure water, and 50% acetonitrile (acetonitrile:water=1:1 water) into 1.5 mL sample vials and position them in slots 61, 62, 63, 64, 65, 66, and 67 of the sample rack of the Shimadzu LC-30AD ultra-high performance liquid chromatograph.

(2) Place 500 μL of the prepared tea infusion or amino acid mixed standard (including 2.5 μmol/mL aspartic acid, 2.5 μmol/mL glutamic acid, 2.5 μmol/mL arginine, 2.5 μmol/mL serine, 2.5 μmol/mL phenylalanine, 2.5 μmol/mL threonine, 2.5 μmol/mL lysine, 2.5 μmol/mL alanine, 2.5 μmol/mL isoleucine, 2.5 μmol/mL tyrosine, 2.5 μmol/mL valine, 2.5 μmol/mL methionine, 2.5 μmol/mL histidine, 2.5 μmol/mL glycine, 2.5 μmol/mL leucine, and 1.25 μmol/mL cysteine) into a 1.5 mL sample vial. Precisely add 50 μL of internal standard (2.5 μmol/mL norvaline), mix well, and place it into the sample rack.

(3) Sample derivatization was performed according to the procedure of Shimadzu Corporation. Chromatographic column: Durashell-AA special column, 3 μm, 4.6×150 mm; Column temperature: 50° C.; Detection wavelengths: primary amino acids at 338 nm, secondary amino acids at 262 nm.

(4) The sample elution procedure was shown in Table 1.

TABLE 1
Amino Acid Composition Analysis Procedure

| Time (min) | Unit | Processing Command | Value |
|---|---|---|---|
| 0.20 | pump | Flow rate (mL/min) | 1.6 |
| 6.00 | pump | Proportion of mobile phase B (%) | 10 |
| 8.00 | pump | Proportion of mobile phase B (%) | 10 |
| 8.00 | pump | Flow rate (mL/min) | 1.6 |
| 10.00 | pump | Proportion of mobile phase B (%) | 16 |
| 10.00 | pump | Flow rate (mL/min) | 1.3 |
| 23:00 | pump | Proportion of mobile phase B (%) | 40 |
| 23:00 | pump | Flow rate (mL/min) | 1 |
| 24.00 | pump | Proportion of mobile phase B (%) | 41 |
| 25.00 | pump | Flow rate (mL/min) | 1.6 |
| 30.00 | pump | Proportion of mobile phase B (%) | 50 |
| 30.00 | pump | Flow rate (mL/min) | 1.6 |
| 31.00 | pump | Proportion of mobile phase B (%) | 100 |
| 34.00 | pump | Proportion of mobile phase B (%) | 100 |
| 35.00 | pump | Proportion of mobile phase B (%) | 5 |
| 35.00 | Auto-sampler | Scavenging | |
| 38.00 | Controller | Stop | |

(5) Based on the concentration and peak area of the internal standard (norvaline) and the peak areas and concentrations of the 16 amino acid standards (aspartic acid, glutamic acid, arginine, serine, phenylalanine, threonine, lysine, alanine, isoleucine, tyrosine, valine, methionine, histidine, cysteine, glycine, and leucine), calculate the content of these 16 amino acids in the sample.

### II. Experimental Results

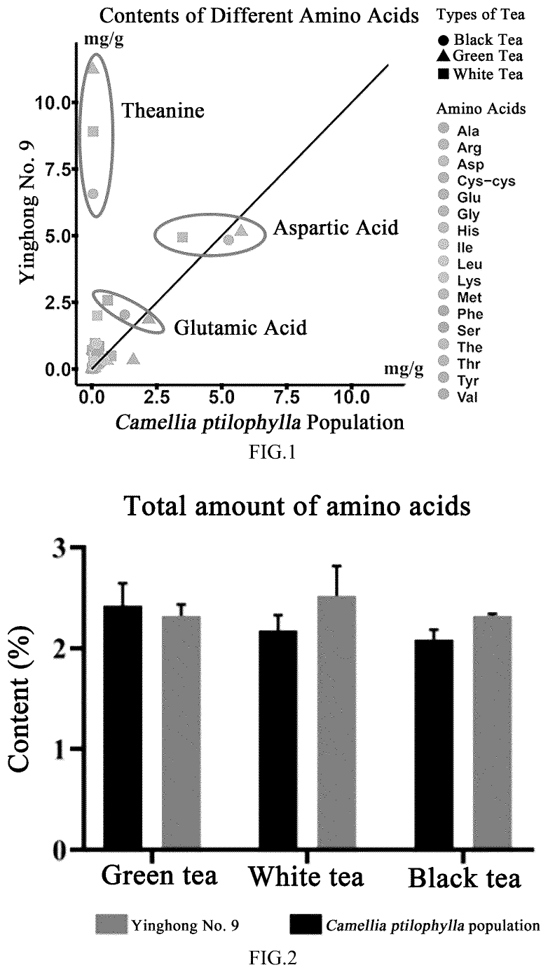

The amino acid components of green tea, white tea, and black tea made from Camellia ptilophylla population and Ying Hong No. 9 were analyzed by OPA derivatization method, and the results were shown in FIG. 1. Theanine is the main free amino acid in green tea, white tea, and black tea of Yinghong No. 9, with contents of 11.90 mg/g, 8.90 mg/g, and 6.54 mg/g, respectively. In contrast, the theanine content in Camellia ptilophylla was significantly lower than that in Yinghong No. 9, with theanine contents of 0.03 mg/g, 0.05 mg/g, and 0.04 mg/g in its green tea, white tea, and black tea, respectively. In addition to theanine, the other major free amino acids in green tea, white tea, and black tea made from Yinghong No. 9 also included aspartic acid and glutamic acid, with their contents showing no significant difference from those in Camellia ptilophylla.

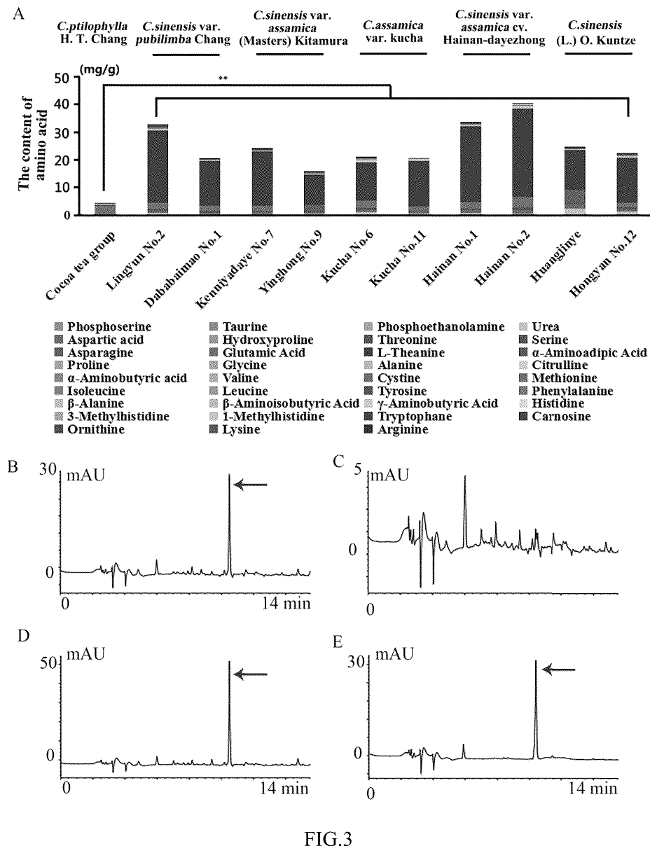

The results of the ninhydrin colorimetric assay showed that the total amino acid contents in green tea, white tea, and black tea made from Camellia ptilophylla population were 2.42%±0.22%, 2.18%±0.15%, and 2.09%±0.09% of the dry matter content, respectively. In contrast, the total amino acid contents in green tea, white tea, and black tea made from Ying Hong No. 9 were 2.32%±0.11%, 2.52%±0.29%, and 2.32%±0.02%, respectively. The analysis of significant differences in p-values revealed no significant difference in the total amino acid content between the green tea, black tea, and white tea made from the Camellia ptilophylla population and Yinghong No. 9 (FIG. 2).

### Example 2: Content of 35 Amino Acids in Steamed Green Samples of Tender Shoots from Camellia ptilophylla Populations and Other 10 Camellia Section Plants

### 1. Experimental Methods

To conduct a more comprehensive analysis of the amino acid composition in Camellia ptilophylla, tender shoots from Camellia ptilophylla populations (mixed from 300 individual plants of Camellia ptilophylla) and 10 non-ptilophylla Camellia section plants were subjected to 35-amino acid composition analysis.

The 10 non-Camellia ptilophylla tea varieties included Lingyun No. 2, Daba Baimao No. 1, Kenya No. 7, Yinghong No. 9, Kucha No. 6, Kucha No. 11, Hainan Daye No. 1, Hainan Daye No. 2, Huangjinye, and Hongyan No. 12. Lingyun No. 2 and Daba Baimao No. 1 belong to the Baimao tea resources, Kenya Daye No. 7 and Yinghong No. 9 belong to the Assam tea resources, Kucha No. 6, Kucha No. 11, Hainan Daye No. 1, and Hainan Daye No. 2 belong to the Assam variety, and Huangjinye and Hongyan No. 12 belong to the Chinese tea resources.

Fresh shoots consisting of one bud and two leaves were collected from 10 Camellia section plants to prepare steamed green tea samples. Additionally, one bud and two leaves from different individual plants of Camellia ptilophylla were mixed together. The 35 amino acid components were analyzed using a fully automatic amino acid analyzer (Sykam S433D, Sykam GmbH, Germany).

The chromatographic conditions employed a 4.6 mm×60 mm column packed with 2622SC cation resin, using a 0.2 mol/L sodium citrate buffer solvent system, with external standard method for quantification of 35 free amino acid components.

### II. Experimental Results

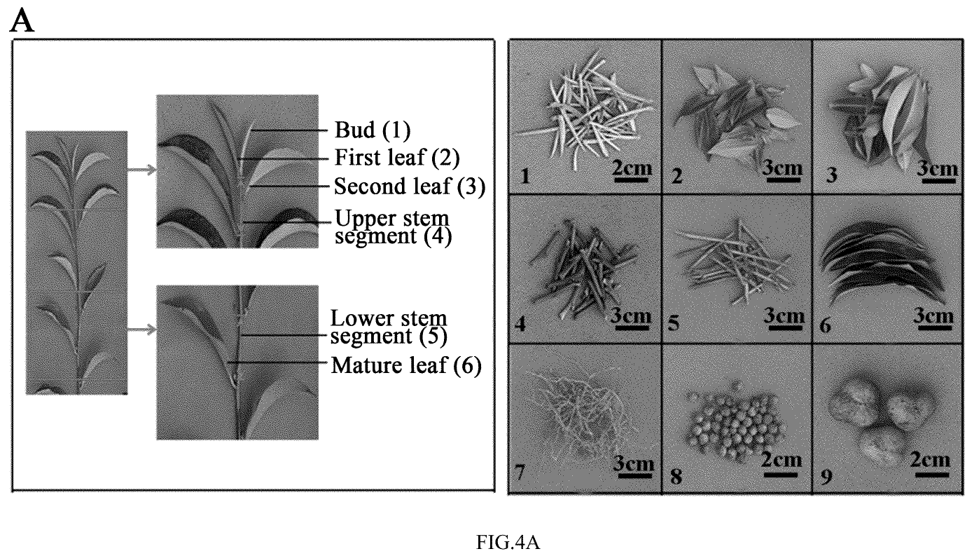

As shown in FIG. 3, 14 amino acids were detected in the tender shoots of the Camellia ptilophylla population. The dry matter content, from highest to lowest, was glutamic acid (2.92 mg/g), aspartic acid (0.55 mg/g), β-aminoisobutyric acid (0.46 mg/g), theanine (0.17 mg/g), arginine (0.05 mg/g), phosphoserine (0.03 mg/g), serine (0.03 mg/g), alanine (0.03 mg/g), tryptophan (0.02 mg/g), threonine (0.02 mg/g), valine (0.02 mg/g), leucine (0.02 mg/g), β-alanine (0.01 mg/g), and Y-aminobutyric acid (0.01 mg/g).

Hydroxyproline, 3-methylhistidine, 1-methylhistidine, carnosine, and α-aminoadipic acid were not detected in the Camellia ptilophylla population or the 10 Camellia section varieties (lines) among the 35 amino acid substances.

Citrulline, asparagine, α-aminobutyric acid, phosphoethanolamine, lysine, taurine, proline, glycine, cystine, methionine, isoleucine, tyrosine, phenylalanine, histidine, and ornithine were detected in some resources of 10 Camellia section plants but were not detected in Camellia ptilophylla.

β-Alanine was only detected in the Camellia ptilophylla population and was not found in the other 10 Camellia section plants. The β-aminoisobutyric acid content in Camellia ptilophylla (0.46 mg/g) was significantly higher than that in the other 10 Camellia section plants (0-0.02 mg/g). The contents of aspartic acid, threonine, serine, and theanine in Camellia ptilophylla were significantly lower than those in other Camellia section plants, especially for theanine. The theanine content in Camellia ptilophylla was only 0.18 mg/g, while the theanine contents in 10 other Camellia section resources were 63-168 times higher than that in Camellia ptilophylla (FIG. 3).

### Example 3: Profile Characteristics of Amino Acid Components in Different Tissue Parts and Distribution of Unknown Amino Acids in Tissues

### 1. Experimental Methods

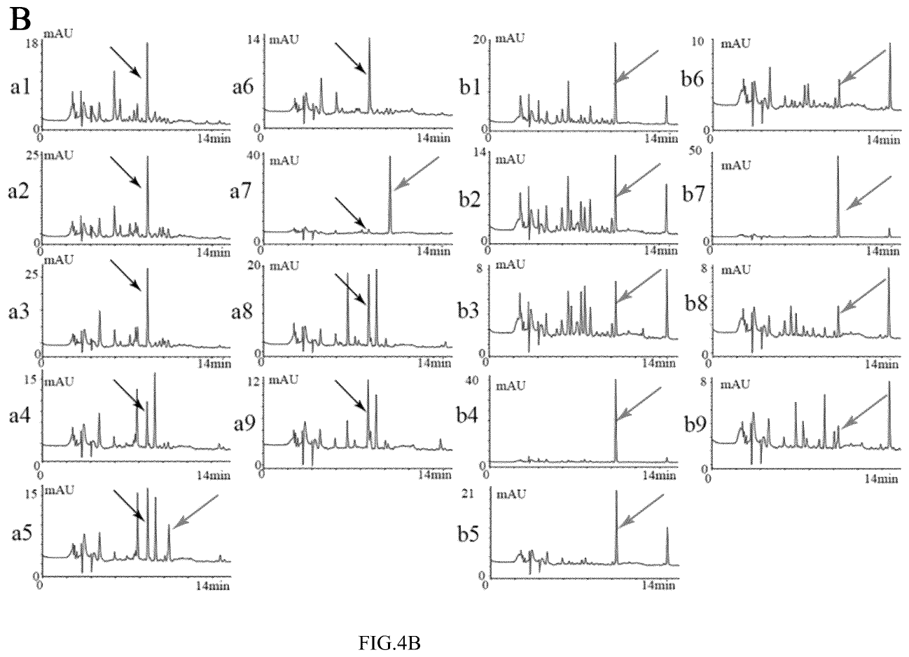

Using the OPA derivatization method, three new strains of Camellia ptilophylla (Camellia ptilophylla No. 1, Camellia ptilophylla No. 2, and Camellia ptilophylla No. 3) and three cultivated tea plant varieties (Zijuan, Yinghong No. 9, and Hongyan No. 12) were selected during spring, summer, and autumn to determine the amino acid composition in different tissue parts (fibrous roots, lower segments of tender stems, upper segments of tender stems, old leaves on semi-lignified stems, the second leaf of tender shoots, the first leaf of tender shoots, and buds). Meanwhile, in summer, the amino acid composition of fruits and flower buds from six varieties (lines) was determined using the OPA derivatization method. The sampling standard was as shown in FIG. 4A. The specific method for determining amino acid composition by the OPA derivatization method was the same as in Example 1.

### II. Experimental Results

There are differences in the chromatograms among the three new strains of Camellia ptilophylla and the three cultivated tea plant varieties. The root chromatogram of Camellia ptilophylla No. 1 resembled that of the cultivated variety Yinghong No. 9, while significant differences were observed in the buds, first leaves, second leaves, old leaves, upper tender stems, fruits, and flower buds (FIG. 4B). The amino acid detection chromatograms from nine tissue parts of Yinghong No. 9 indicated that theanine is the predominant free amino acid in the cultivated tea plant varieties, with its peak appearing at approximately 10.4 min. The amino acid detection chromatogram of the aerial tissues of Camellia ptilophylla showed no significant peak at the 10.4-minute mark, but a relatively distinct peak was observed at 7.6 min. This peak differs from the retention times of the 16 common amino acid standards found in tea. This peak is the product of a reaction with the derivative reagent OPA (o-phthalaldehyde), indicating that the substance may be an uncommon amino acid or a novel amino acid (unknown amino acid) specific to Camellia ptilophylla.

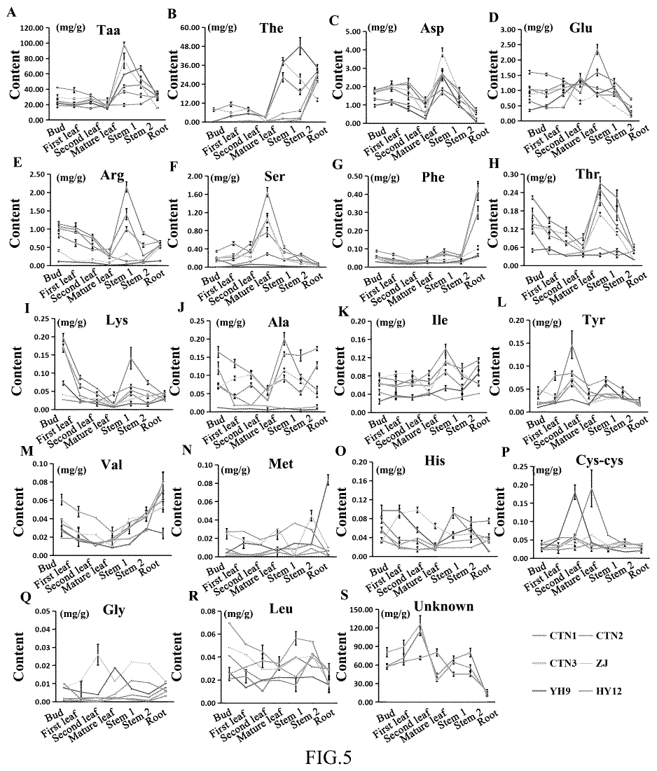

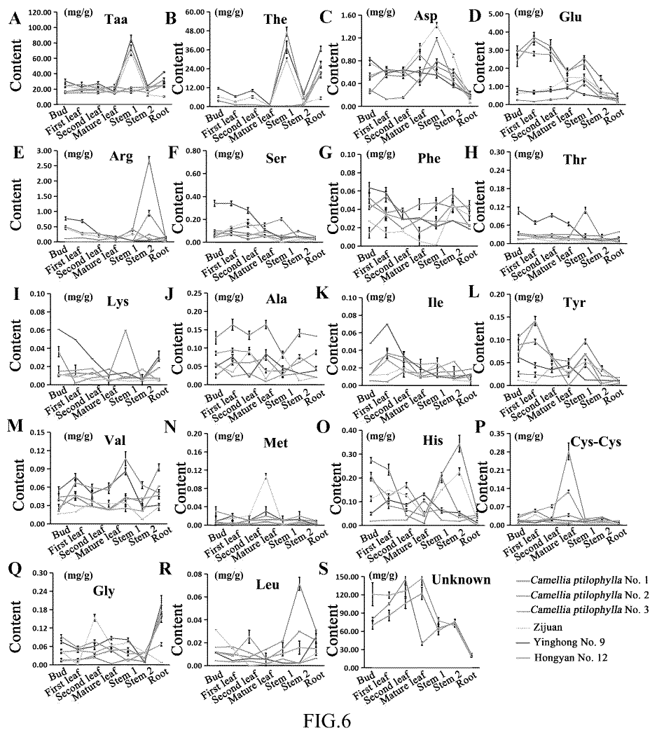

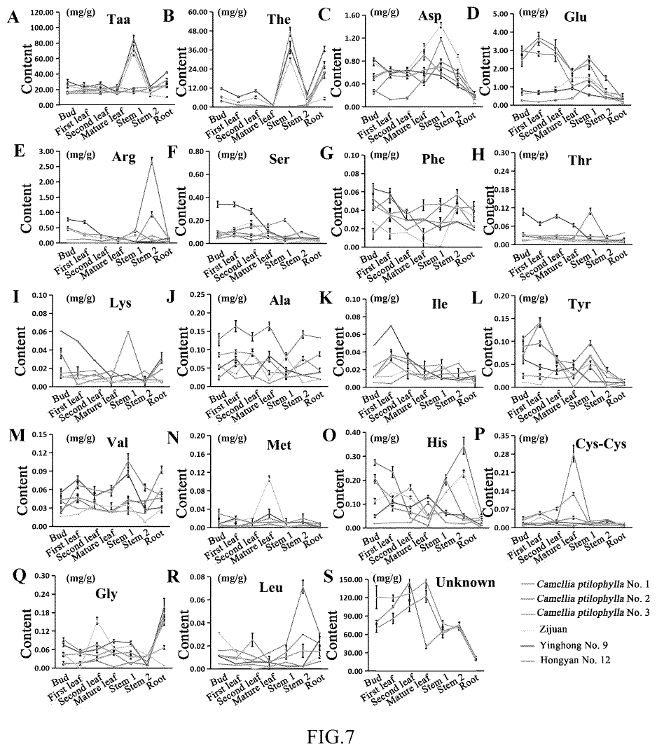

By measuring the amino acid composition in different tissue parts during the three seasons of spring (Panels A-S of FIG. 5), summer (Panels A-S of FIG. 6), and autumn (Panels A-S of FIG. 7) and analyzing the major free amino acids, it was found that theanine (Panel B of FIG. 5 for spring, Panel B of FIG. 6 for summer, Panel B of FIG. 7 for autumn), asparagine (Panel C of FIG. 5 for spring, Panel C of FIG. 6 for summer, Panel C of FIG. 7 for autumn), and glutamic acid (Panel D of FIG. 5 for spring, Panel D of FIG. 6 for summer, Panel D of FIG. 7 for autumn) were the main free amino acids in the three cultivated varieties. In contrast, the most abundant amino acids in Camellia ptilophylla were an unknown amino acid (Panel S of FIG. 5 for spring, Panel S of FIG. 6 for summer, Panel S of FIG. 7 for autumn), glutamic acid (Panel D of FIG. 5 for spring, FIG. 6 for summer, FIG. 7 for autumn), and asparagine (Panel C of FIG. 5 for spring, Panel C of FIG. 6 for summer, Panel C of FIG. 7 for autumn).

The detection revealed that this unknown amino acid was specifically present in Camellia ptilophylla and was not detected in any tissues of the three cultivated tea plant varieties. Trace amounts of the unknown amino acid from Example 3 were detected in the root systems of Camellia ptilophylla, with higher levels found in the tender shoots, the highest content of this unknown amino acid in autumn was observed in the second leaves of Camellia ptilophylla No. 1 and Camellia ptilophylla No. 3, whereas Camellia ptilophylla No. 2 showed the highest content in old leaves (Panel S of FIG. 5 for spring, Panel S of FIG. 6 for summer, Panel S of FIG. 7 for autumn).

### Example 4: Correlation Analysis of Unknown Amino Acids with Total Amino Acids, Theanine, and Glutamic Acid

### I. Experimental Methods

The detection method and detection data were the same as those in Example 3.

### II. Experimental Results

Since theanine and glutamic acid are related in the metabolic pathway, and the unknown amino acid may be associated with theanine, the focus was on comparing the correlations among these three.

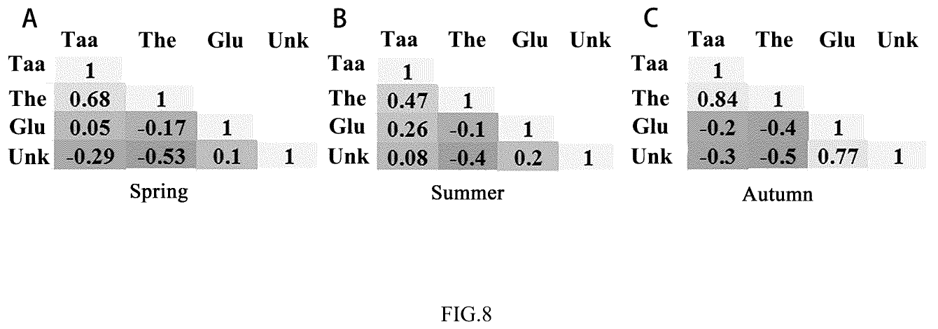

The results showed that the total amino acid content and amino acid components varied among different tea plant varieties and tissue parts across different seasons (Panels A-S of FIG. 5 for spring, Panels A-S of FIG. 6 for summer, and Panels A-S of FIG. 7 for autumn). Based on the amino acid composition of different varieties and tissues in spring, summer, and autumn, a correlation analysis was conducted between total amino acids and major free amino acids (theanine, glutamic acid, and the unknown amino acid from Example 3). The results showed that there was a certain correlation among total amino acids, theanine, the unknown amino acid from Example 3, and glutamic acid across the three seasons. Theanine showed a high correlation with both total amino acids and the unknown amino acid in Example 3 across all three seasons (FIG. 8). The correlation coefficients between theanine and total amino acids for the three seasons were 0.68, 0.47, and 0.84, respectively. The correlation coefficients between theanine and the unknown amino acid in Example 3 for the three seasons were −0.53, −0.4, and −0.5, respectively. The correlation between theanine and glutamic acid was relatively low in spring and summer, with coefficients of −0.17 and −0.1 (Panels A and B of FIG. 8), while it was relatively higher in autumn, with a coefficient of −0.4 (Panel C of FIG. 8). In Example 3, the unknown amino acid exhibited relatively low correlation with glutamic acid in spring and summer, with correlation coefficients of 0.1 and 0.2, respectively, while showing a relatively high correlation in autumn, with a coefficient of 0.77. The correlation between the unknown amino acids in Example 3 and the total amino acids in spring, summer, and autumn was −0.29, 0.08, and −0.30, respectively.

Therefore, the unknown amino acid in Example 3 is an amino acid that exhibits a highly negative correlation with theanine.

### Example 5: Isolation and Identification of Unknown Amino Acid from Example 3 in Camellia ptilophylla

### I. Isolation of Unknown Amino Acids in Example 3 of Camellia ptilophylla

The OPA derivatization method from Example 1 was used to determine the contents of unknown amino acids and theanine in 300 individual plants of Camellia ptilophylla and 100 other Camellia section plants from Example 3. The separation and extraction of amino acids required the derivatization of the extract from the steamed green leaves of Camellia ptilophylla with DNFB (2,4-dinitrofluorobenzene). From 300 individual plants of Camellia ptilophylla, two individual plants with high unknown amino acid content and two with low unknown amino acid content were selected for amino acid detection using the DNFB (2,4-dinitrofluorobenzene) derivatization method. By comparing the peak shapes, it was determined that the unknown amino acid in Example 3 had a peak time at 42 min in the DNFB (2,4-dinitrofluorobenzene) derivatization method.

### 1. DNFB Extensively Derivatized Amino Acids

### (1) Experimental Method

The steamed green tea sample of the Camellia ptilophylla population was extracted at a ratio of 4 L of 100° C. water per 1 kg of steamed green sample, with an extraction time of 2 h. The sample was initially filtered through two layers of gauze after extraction to remove tea residues and obtain the primary extract. The primary extract was centrifuged at 3,500 rpm, and the supernatant was collected as the tea infusion.

The DNFB derivatization method for amino acid separation and extraction (large-scale derivatization method) was as follows: dilute the prepared 8 L of tea infusion by 5 times, measure 300 mL of the tea infusion into a 1 L beaker, then add 300 mL of 10 mL/L DNFB acetonitrile solution and 30 mL of 0.5 M NaHCO₃ buffer solution (pH 9.0). Mix well and derivatize in a 60° C. water bath in the dark for 1 h to obtain the derivatized product. The tea infusion was derivatized through multiple reactions, and all derivatized products were concentrated to 20 L (the product of large-scale derivatization of tea infusion with DNFB) for subsequent separation and purification.

The tea infusion was subjected to HPLC analysis after extensive derivatization with DNFB to observe the peak at 42 min, aiming to determine whether the unknown amino acid from Example 3 was present in the DNFB (2,4-dinitrofluorobenzene) derivatization products of the tea infusion. The specific HPLC analysis method was as follows:

Liquid chromatograph (Agilent 1200), column (Eclipse Plus C18, 250 mm×4.6 mm, 5 μm).

The mobile phase A for liquid chromatography analysis was a 0.05 M NaAc buffer solution (pH 6.5, containing 10 mL/L N,N-dimethylformamide), and the mobile phase B was an acetonitrile aqueous solution (V/V=1:1).

The mobile phase flow rate was 1.0 mL/min, and the gradient elution program was shown in Table 2.

Column temperature: 28° C.; Sample injection volume: 5 μL; the instrument detection wavelength: 360 nm.

TABLE 2
Elution program for HPLC analysis of DNFB-
extensively derivatized amino acids

| Time (min) | A (%) | B (%) |
|---|---|---|
| 0 | 100 | 0 |
| 6 | 90 | 10 |
| 12 | 85 | 15 |
| 18 | 80 | 20 |
| 33 | 75 | 25 |
| 42 | 70 | 30 |
| 50 | 50 | 50 |
| 55 | 20 | 80 |
| 60 | 0 | 100 |
| 65 | 0 | 100 |
| 70 | 100 | 0 |
| 80 | 100 | 0 |

### (2) Experimental Results

The derivatized product was analyzed by HPLC, which showed a distinct peak at 42 min, indicating successful derivatization.

### 2. First Purification

Take 20 L of the tea infusion product after large-scale derivatization with DNFB, adjust the pH to approximately 3.0 with phosphoric acid, remove the organic solvent by rotary evaporation, and load it onto a 100×500 mm HP-20 macroporous resin column. Elute sequentially with 10% (V/V) acetonitrile aqueous solution, 30% (V/V) acetonitrile aqueous solution, 50% (V/V) acetonitrile aqueous solution, 70% (V/V) acetonitrile aqueous solution, and 90% (V/V) acetonitrile aqueous solution, each for 30 min. Monitor the eluate by HPLC, and combine the eluate corresponding to the target peak at 42 min.

The obtained eluate containing the target peak was evaporated to dryness, dissolved in 2 L of a 10% (V/V) acetonitrile aqueous solution, and filtered to remove the filter residue, yielding the crude separated extract. The crude extract was subjected to a second purification using preparative liquid chromatography (Hanbon High-Pressure Preparative Chromatography DAC100).

### 3. Second Purification

The chromatographic conditions for secondary purification were as follows: Chromatographic column (DAC column, 100×250 nm, 10 μm), packing material (YMC C18), wavelength of 360 nm, flow rate of 80 mL/min.

The mobile phase solution system for secondary purification was as follows: Mobile phase A was acetonitrile, and mobile phase B was an aqueous solution of 0.1% acetic acid, calculated by volume. The gradient elution program was as follows: 0 min-10% A, 40 min-10% A, 100 min-15% A, 140 min-20% A, 170 min-25% A, 195 min-25% A, 200 min-90% A, 220 min-90% A.

The collected solution was analyzed by HPLC, and the fractions from Example 3 with unknown amino acid purity greater than 90% were combined.

### 4. The Third Purification

The collected solution obtained after the second purification was combined, diluted with purified water by half, and then pumped for loading to proceed with the third purification.

The chromatographic conditions and mobile phase solution system for the third purification were the same as those for the second purification.

The gradient elution program by volume was as follows: 0 min-15% A, 5 min-15% A, 15 min-30% A, 25 min-30% A, 40 min-50% A.

Analyze the collected fractions by HPLC, and combine those with unknown amino acid purity greater than 95%.

The collected solution was rotary evaporated to remove acetonitrile, followed by freeze-drying to obtain the target compound.

### 5. Nuclear Magnetic Resonance (NMR) Detection of the Target Substance (Unknown Amino Acid Derivative from Example 3)

### (1) Experimental Method

By employing 600 MHz nuclear magnetic resonance (NMR), the target compound (the unknown amino acid derivative from Example 3) was analyzed to obtain 1H NMR, COSY, HSQC, DEPT, 13C NMR, and HMBC spectra. Based on the 1H-NMR spectrum combined with COSY, HSQC, and DEPT spectra, the 1H spectral signals of the compound were assigned.

The obtained target compound (the unknown amino acid derivative from Example 3) was dissolved in an acetonitrile solution and subjected to GC-MS/MS detection on an ultra-high-resolution liquid chromatography-mass spectrometer, with analysis performed in positive ion mode of the first-order mass spectrometry.

### (2) Experimental Results

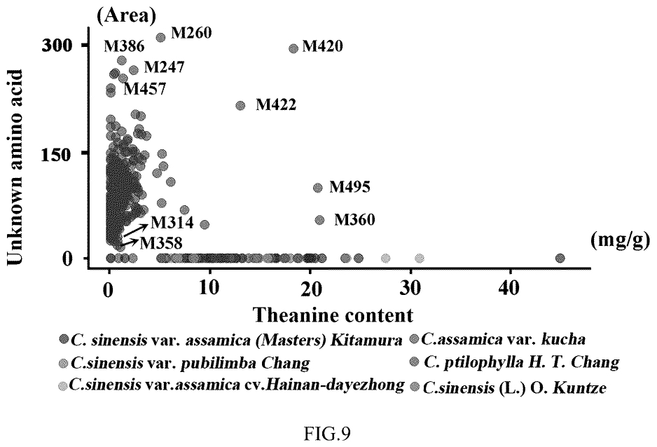

The detection of unknown amino acids in 300 individual plants of Camellia ptilophylla using the OPA method revealed that all 300 Camellia ptilophylla resources contained the unknown amino acids described in Example 3, while none were detected in the other 100 resources (partial results were shown in FIG. 9). The peak areas of unknown amino acids in Example 3 for the individual plants M260, M386, and M420 from the Camellia ptilophylla population were relatively large, measuring 314.1, 281.3, and 298.2, respectively. In contrast, the peak areas of unknown amino acids in Example 3 for the individual plants M314, M338, and M598 were relatively small, measuring 24.80, 28.70, and 18.60, respectively. The detection results indicated that the unknown amino acids from Example 3 was detected in all 300 samples of Camellia ptilophylla, but was absent in all 100 samples of other Thea section plants. The content of the unknown amino acids in Example 3 varied among different individual plants of Camellia ptilophylla. The peak positions of the unknown amino acid in Example 3 in the DNFB derivatization method can be determined by using three Camellia ptilophylla individuals with high and three with low contents of the unknown amino acid in Example 3.

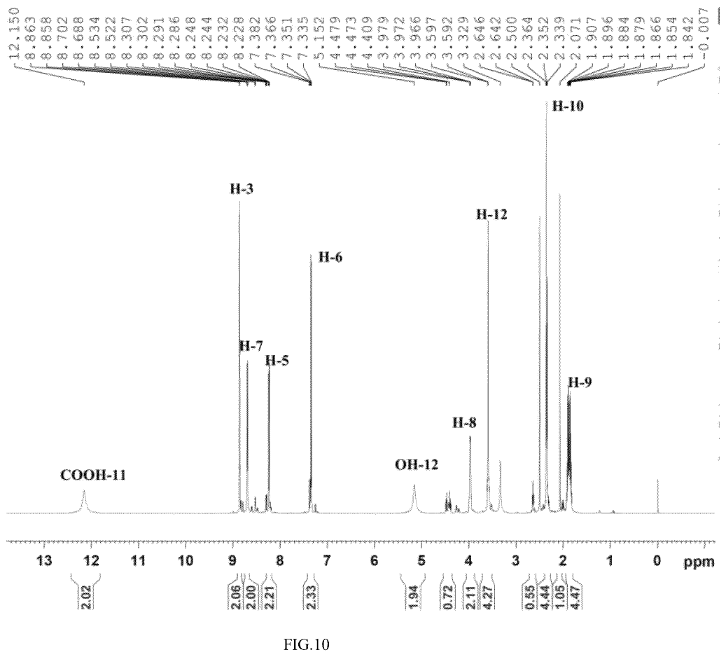

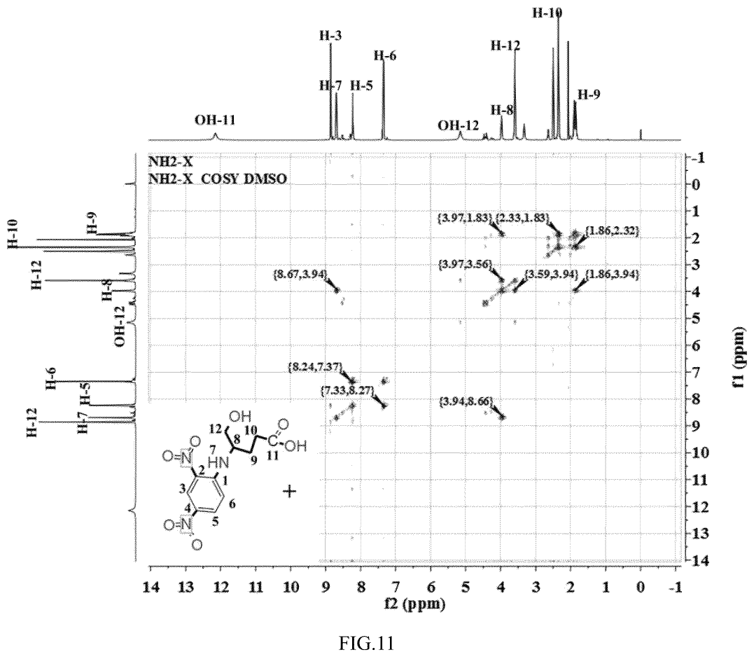

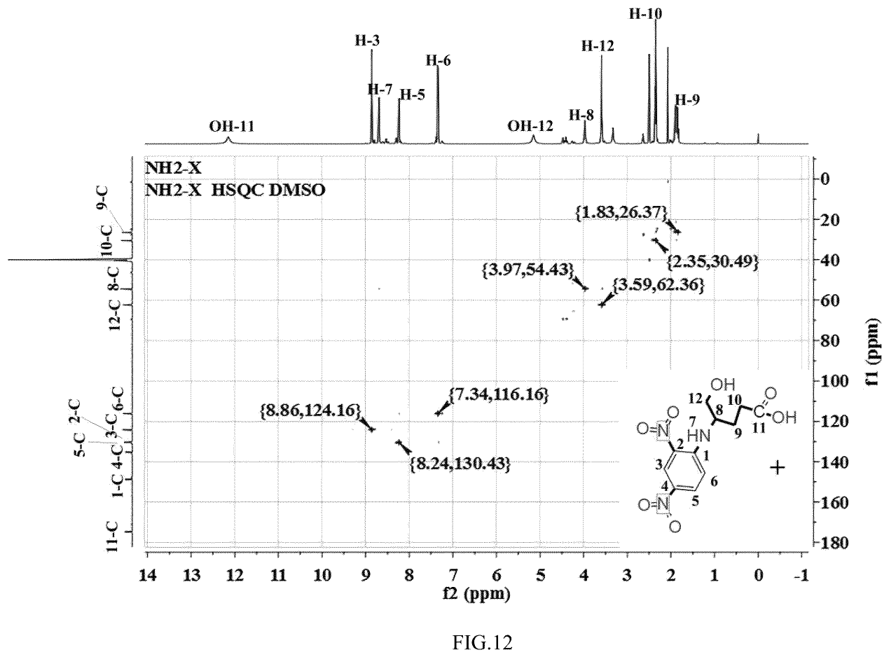

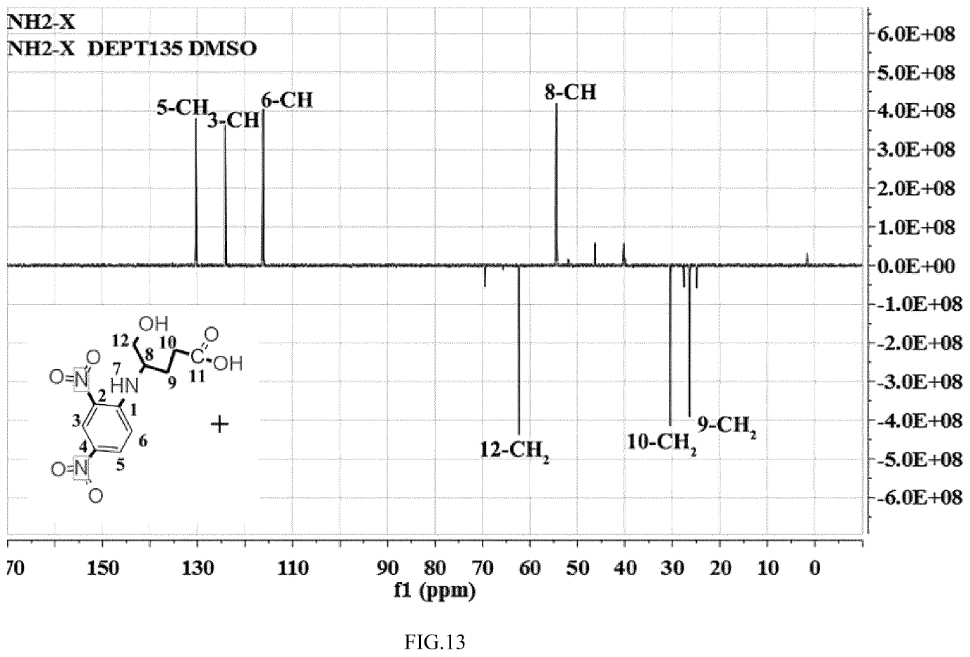

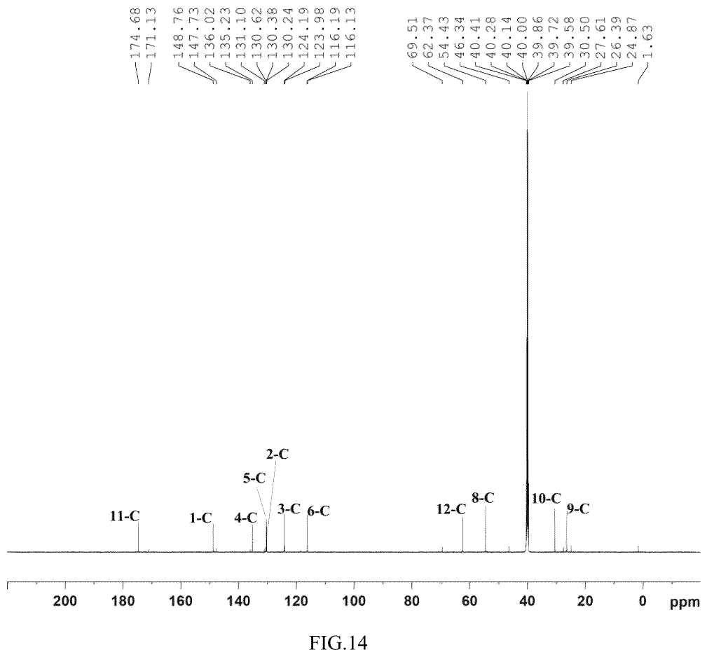

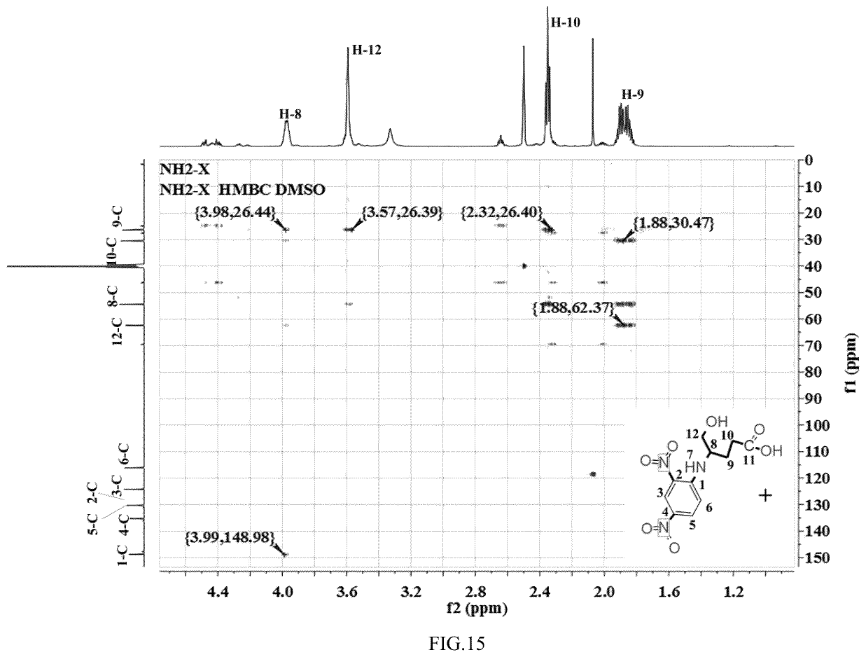

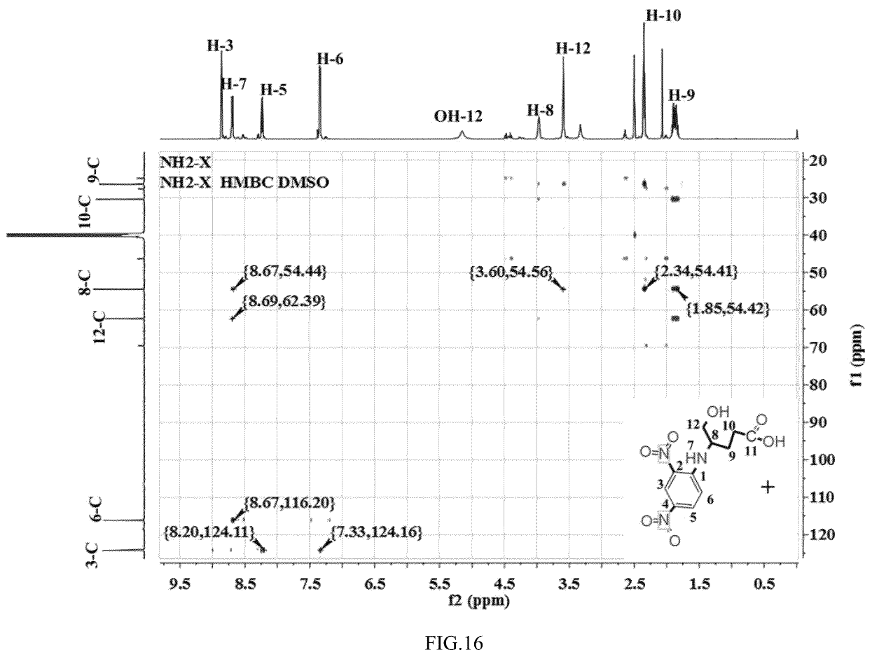

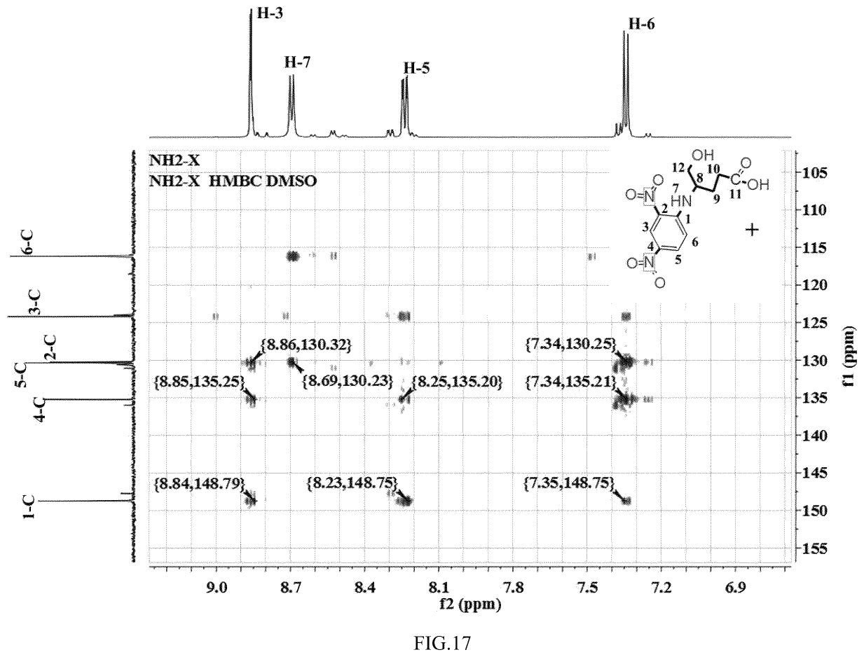

The hydrogen spectrum (¹H-NMR) of the unknown amino acid derivative in Example 3 is shown in FIG. 10, with the COSY spectrum in FIG. 11, the HSQC spectrum in FIG. 12, the DEPT spectrum in FIG. 13, the carbon spectrum (¹³C-NMR) in FIG. 14, and the HMBC spectra in FIG. 15, FIG. 16, and FIG. 17.

The ¹H-NMR shows 10 sets of hydrogens, with integration ratios from downfield to upfield being 1:1:1:1:1:1:1:2:2:2, corresponding to 13 protons in the molecular structure of the compound. At δ12.150, the hydrogen appeared as a broad singlet with a proton count of 1. The COSY spectrum showed no correlation with other hydrogen protons, while HSQC and DEPT spectra indicated no carbon correlation for this hydrogen, which was assigned as COOH-11. At 88.860, the hydrogen appeared as a doublet with a proton count of 1. The COSY spectrum showed no correlation with hydrogen protons, while the HSQC and DEPT spectra indicated that this hydrogen was correlated with the methylene carbon (δ 124.19), and thus assigned as H-3. At δ 8.695, the hydrogen appeared as a doublet with a proton count of 1. The COSY spectrum indicated correlation with the hydrogen proton at δ 3.972. HSQC and DEPT spectra showed no carbon correlation for this hydrogen, which was assigned as H-7. At δ 8.238, the hydrogen appeared as a dd peak with a proton count of 1. The COSY spectrum indicated correlation with the hydrogen proton at δ 7.343. HSQC and DEPT spectra showed that this hydrogen was correlated with a methine carbon (δ 130.38), and thus assigned as H-5. At δ 7.343, the hydrogen appeared as a doublet with a proton count of 1. The COSY spectrum indicated its correlation with the hydrogen proton at δ 8.238. HSQC and DEPT spectra showed that this hydrogen was correlated with a methine carbon (δ 116.19), and thus assigned as H-6. At δ5.152, the hydrogen appeared as a broad singlet with a proton count of 1. The COSY spectrum showed correlation with the hydrogen proton at 83.592, while HSQC and DEPT indicated no carbon correlation for this hydrogen, which was assigned as OH-12. The hydrogen at δ 3.972 appeared as a multiplet with 1 proton. The COSY spectrum showed correlations with hydrogen protons at δ 8.695, δ 3.593, and δ 1.872. HSQC and DEPT indicated that this hydrogen was connected to a methine carbon (δ 54.43) and was assigned as H-8. At δ 3.592, the hydrogen appeared as a multiplet with a proton count of 2. The COSY spectrum indicated correlation with hydrogen protons at δ 3.972 and δ 5.152. HSQC and DEPT spectra showed that this hydrogen was correlated with a methylene carbon (δ 62.38), thus assigned as H-12. The hydrogen at δ 2.352 appeared as a triplet with a proton count of 2. The COSY spectrum indicated its correlation with the hydrogen proton at δ 1.872, while HSQC and DEPT spectra showed its correlation with a methylene carbon (δ 30.50), thus assigned as H-10. The hydrogen at δ 1.872 appeared as a multiplet with 2 protons. The COSY spectrum showed correlations with hydrogen protons at δ 2.352 and δ 3.972. HSQC and DEPT spectra indicated that this hydrogen was connected to a methylene carbon (δ 26.40), and thus assigned as H-9.

Based on the ¹³C-NMR spectrum, combined with HSQC, DEPT, and HMBC spectra, the ¹³C spectrum of the compound can be assigned. The DEPT spectrum and two-dimensional spectra assisted in the interpretation of the carbon spectrum as follows: there are a total of 11 effective peaks, including 3 secondary carbon peaks, 4 tertiary carbon peaks, and 4 quaternary carbon peaks.

δ26.40, δ30.50, and δ62.38 were secondary carbon signals; the HSQC spectrum showed a correlation between δ26.40 and the hydrogen proton at δ1.872, while the HMBC spectrum (FIG. 15) indicated correlations with hydrogen protons at δ8.695, δ3.972, δ3.592, and δ2.352, thus assigning it to C-9. The HSQC spectrum indicated a correlation between δ30.50 and the hydrogen proton at δ2.352, while the HMBC spectrum (FIG. 15) showed correlations with the hydrogen protons at δ3.972 and δ1.872, thus assigning it to C-10. The HSQC spectrum indicated a correlation between δ62.38 and the hydrogen proton at δ3.592, while the HMBC spectrum (FIG. 15) showed correlations with hydrogen protons at δ8.695, δ3.972, and δ1.872, thus assigning it to C-12.

δ54.43, δ116.19, δ124.19, and δ130.38 were signals of four tertiary carbons. The HSQC spectrum indicated a correlation between δ54.43 and the hydrogen proton at δ3.972, while the HMBC spectrum (FIG. 16) showed correlations with hydrogen protons at δ8.695, δ3.592, δ2.352, and δ1.872, thus assigning it to C-8. The HSQC spectrum indicated a correlation between δ116.19 and the hydrogen proton at δ7.343, while the HMBC spectrum (FIG. 16) showed a long-range correlation with δ8.695, thus assigning it to C-6 on the benzene ring. The HSQC spectrum indicated a correlation between δ124.19 and the hydrogen proton at δ8.860, while the HMBC spectrum (FIG. 16) showed long-range correlations with δ8.238 and δ7.343, thus assigning it to C-3 on the benzene ring. The HSQC spectrum indicated a correlation between δ130.38 and the hydrogen proton at δ8.238, while the HMBC spectrum (FIG. 17) showed long-range correlations with δ8.860 and δ7.343, thus assigning it to C-5 on the benzene ring.

The signals at δ130.25, δ135.23, δ148.76, and δ174.68 corresponded to four quaternary carbon signals. The HMBC spectrum (FIG. 17) showed long-range correlations between δ 130.25 and δ 8.860, δ8.695, δ7.343, thus assigning it to the benzene ring C-2. The HMBC spectrum (FIG. 17) showed that δ135.23 has long-range correlations with δ8.860, δ8.238, and δ7.343, thus being assigned to the benzene ring C-4. The HMBC spectrum (FIG. 17) showed that δ148.76 has long-range correlations with δ8.860, δ8.238, δ7.343, and δ3.972, thus being assigned to the benzene ring C-1. The HMBC spectrum (FIG. 17) showed long-range correlations between δ174.68 and δ2.352, δ1.872, thus assigning it to C-11.

Therefore, the structural formula of the unknown amino acid is as shown in Formula (I).

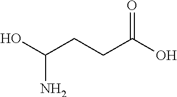

6. Mass spectrometry analysis of the target compound (unknown amino acid derivative from Example 3).

### (1) Experimental Method

The obtained target compound (the unknown amino acid derivative from Example 3) was analyzed on the ultra-high-resolution liquid chromatography-mass spectrometry. The mass spectrometry conditions were as follows: Chromatographic column (ACQUITY UPLC BEH C18, 2.1×100 mm, 1.7 μm).

The mobile phase solution system was as follows: Mobile phase A is 0.1% formic acid, and mobile phase B is acetonitrile.

The elution procedure was shown in Table 3:0 min-90% A, 10 min-5% A, 15 min-5% A, 15.1 min-90% A, 20 min-90% A, with a flow rate of 0.3 mL/min.

The secondary fragments of ionization were analyzed using Mass Frontier software.

TABLE 3
High-resolution mass spectrometry analysis of the elution
system for unknown amino acid derivatives in Example 3.

| Time (min) | A (%) | B (%) |
|---|---|---|
| 0 | 90 | 10 |
| 10 | 5 | 95 |
| 15 | 5 | 95 |
| 15.1 | 90 | 10 |
| 20 | 90 | 10 |

### (2) Experimental Results

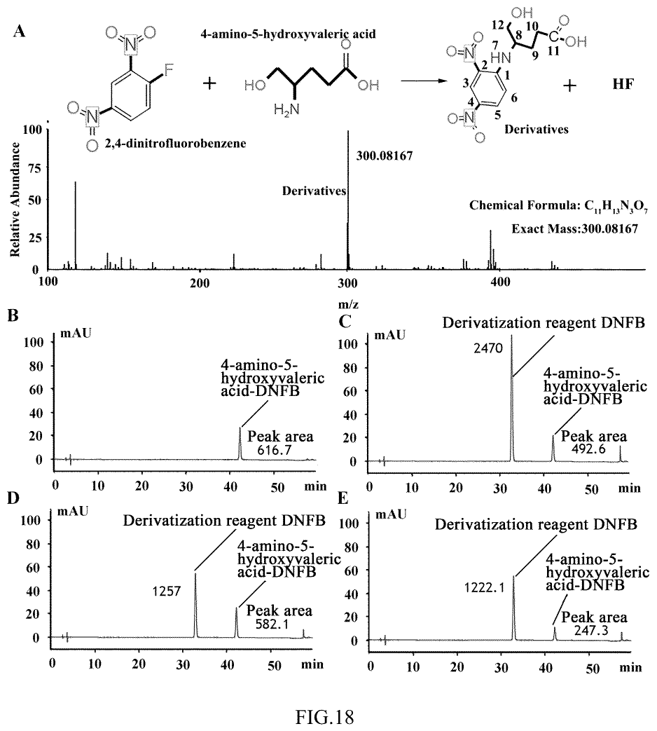

The primary mass spectrometry results indicated that the molecular weight of the unknown amino acid derivative in Example 3 is 300.1. Through the chemical reaction equation of amino acids with the DNFB derivatization reagent, the chemical formula of the DNFB derivatization reagent, the chemical structure identified by NMR analysis, and the molecular weight determined by primary mass spectrometry, the unknown amino acid in Example 3 was ultimately confirmed to be 4-amino-5-hydroxypentanoic acid (Panel A of FIG. 18).

### 7. Verification of the Structural Formula of 4-Amino-5-Hydroxypentanoic Acid

### (1) Experimental Method

The derivative of the unknown amino acid from Example 3 obtained in the previous step of separation and purification (i.e., the DNFB derivative of 4-amino-5-hydroxypentanoic acid, denoted as DNFB-4-amino-5-hydroxypentanoic acid) was used as Sample 1. Chemically synthesize the standard of 4-amino-5-hydroxypentanoic acid, and perform DNFB derivatization according to the small-scale derivatization method, with the derivative product serving as sample 2. Sample 1 and Sample 2 were subjected to HPLC analysis separately.

Additionally, mix the solutions of Sample 1 and Sample 2 in equal volumes to prepare Sample 3, and then perform HPLC detection.

Mix sample 2 and water in equal volumes to prepare sample 4, then perform HPLC analysis.

The HPLC analysis method was the same as the DNFB derivative analysis conditions in Example 5.

The specific methods for a small number of derivative methods were as follows: take a 25 mL volumetric flask, add 2.0 mL of tea infusion, then add 2.0 mL of 10 mL/L DNFB acetonitrile solution, followed by 2 mL of 0.5 M pH 9.0 NaHCO₃ buffer solution. Perform light-protected derivatization in a 60° C. water bath for 1 h. After cooling, the solution was diluted to volume with 0.01M KH₂PO₄ buffer solution (pH 7.0), shaken well, filtered through a 0.45 μm filter, and subjected to HPLC analysis.

The specific HPLC analysis method was as follows:

Liquid chromatograph (Agilent 1200), column (Eclipse Plus C18, 250 mm×4.6 mm, 5 μm).

The mobile phase A for liquid chromatography analysis was a 0.05 M NaAc buffer solution (pH 6.5, containing 10 mL/L N,N-dimethylformamide), and the mobile phase B was an acetonitrile aqueous solution (V/V=1:1).

The mobile phase flow rate was 1.0 mL/min, and the gradient elution program was shown in Table 2.

Column temperature: 28° C.; Sample injection volume: 5 μL; the instrument detection wavelength was 360 nm.

### (2) Experimental Results

It was found that the elution times of Sample 1 and Sample 2 were identical (Panels B and C of FIG. 18), both peaking at approximately 42 min.

Additionally, the solutions of Sample 1 and Sample 2 were mixed in equal volumes and subjected to HPLC analysis. The mixed sample exhibited a single peak at 42 min, with its peak area approximating the average of those of Sample 1 and Sample 2 (Panel D of FIG. 18).

The sample 2 was mixed with water in equal volumes and then subjected to HPLC analysis, showing a peak area at 42 min that was approximately half of that in sample 2 (Panel E of FIG. 18).

The experimental results from FIGS. 18B to 18E demonstrated the accuracy of nuclear magnetic resonance (NMR) and mass spectrometry (MS) in identifying the structure of 4-amino-5-hydroxypentanoic acid.

Finally, it should be noted that the above embodiments are only intended to illustrate the technical solutions of the present disclosure and not to limit the scope of protection of the present disclosure. For those skilled in the art, other variations or modifications in different forms may be made based on the above description and concepts. It is neither necessary nor possible to exhaustively list all possible implementations here. Any modifications, equivalent replacements, and improvements made within the spirit and principles of the present disclosure shall be included within 10 the scope of protection of the claims of the present disclosure.

What is claimed is:

1. A compound having a structural formula of Formula (I),

| - | |
|---|---|
| | OH |
| | NH? |

(I) .

2. (canceled)

3. A method for identifying Camellia ptilophylla, comprising detecting whether the compound according to claim 1 is present in a sample to be tested;
the sample to be tested is Camellia ptilophylla under the condition that the sample to be tested contains the compound according to claim 1.

4. A method for detecting a compound having a structural formula of Formula (I), comprising performing a high-performance liquid chromatography (HPLC) analysis on a 2,4-dinitrofluorobenzene (DNFB) derivatization product of an aqueous extract of the sample to be tested.

5. The method according to claim 4, wherein a chromatographic column is an Eclipse Plus C18 column, and a mobile phase comprises mobile phase A and mobile phase B, and wherein the mobile phase A is a NaAc buffer solution containing N,N-dimethylformamide, the mobile phase B is an acetonitrile aqueous solution, and a gradient elution is performed.

6. (canceled)

7. A kit for detecting and identifying Camellia ptilophylla, comprising a detection reagent for the compound according to claim 1.

8. The kit according to claim 7, wherein the detection reagent is a derivatization reagent and/or an HPLC analysis reagent.

9. The kit according to claim 8, wherein the derivatization reagent is an acetonitrile solution of DNFB and a NaHCO₃ buffer solution.

10. The kit according to claim 8, wherein the HPLC analysis reagent comprises a mobile phase, the mobile phase comprises mobile phase A and mobile phase B, and wherein the mobile phase A is a NaAc buffer solution comprising N,N-dimethylformamide, and the mobile phase B is an acetonitrile aqueous solution.

11. The method according to claim 3, wherein the sample to be tested is tender shoots of Camellia ptilophylla.

12. The method according to claim 3, wherein a high-performance liquid chromatography analysis is performed on a 2,4-dinitrofluorobenzene derivatization product of an aqueous extract of the sample to be tested.

13. The method according to claim 12, wherein the aqueous extract of the sample to be tested is prepared as follows: the sample to be tested is mixed with boiling water, fully extracted under a boiling condition, and a solid is removed to obtain the aqueous extract.

14. The method according to claim 13, wherein the sample to be tested is in powder form, and a ratio of the sample to be tested to the aqueous extract is 1 g: 80 mL to 120 mL, with an extraction time of 30 min to 50 min.

15. The method according to claim 13, wherein the sample to be tested is a steamed green sample, and a ratio of the sample to be tested to the aqueous extract is 1 kg: 3 L to 5 L, with the extraction time of 1 h to 3 h.

16. The method according to claim 12, wherein the 2,4-dinitrofluorobenzene derivatization product is obtained as follows: a mixture of the aqueous extract of the sample to be tested, an acetonitrile solution of 2,4-dinitrofluorobenzene and a NaHCO₃ buffer solution undergoes a derivatization reaction in the dark at 50° C. to 70° C.

17. The method according to claim 16, wherein the mixture is a combination of the aqueous extract of the sample to be test, an 8 mL/L to 12 mL/L acetonitrile solution of 2,4-dinitrofluorobenzene, and a 0.4 mol/L to 0.6 mol/L NaHCO₃ buffer solution at a volume ratio of 1 to 10:1 to 10:1 to 1.

18. The method according to claim 3, wherein a chromatographic column is an Eclipse Plus C18 column, and a mobile phase comprises mobile phase A and mobile phase B, and wherein the mobile phase A is a NaAc buffer solution containing N,N-dimethylformamide, the mobile phase B is an acetonitrile aqueous solution, and a gradient elution is performed.

19. The method according to claim 18, wherein a temperature of the chromatographic column is 26° C. to 30° C.

20. The method according to claim 12, wherein a detection wavelength is 300 nm to 420 nm in the high-performance liquid chromatography analysis.
